(19) United States
(12) Patent Application Publication
(10) Pub. No.: US 2007/0282294 A1
(43) Pub. Date: Dec. 6, 2007

(54) Ampoule and Administering Device

(75) Inventor: Rudolf Sidler, Langendorf (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Disetronic Licensing AG

(21) Appl. No.: 11/737,834

(22) Filed: Apr. 20, 2007

Related U.S. Application Data
(63) Continuation of application No. 10/443,246, filed on May 22, 2003, now abandoned.

(30) Foreign Application Priority Data
May 24, 2002 (DE) 102 23 192.3

(51) Int. Cl. A61M 5/28

(52) U.S. Cl. 604/407; 604/403

(57) ABSTRACT

An ampoule for containing an injectable substance including an ampoule body and a component of a means for injecting, the component carried by the ampoule body, wherein other components of the means for injecting are associated with an administering device to which the ampoule can be coupled and are operably coupleable to the component carried by the ampoule body. A method of administering an injectable substance using the ampoule is encompassed.

### CROSS-REFERENCE TO RELATED APPLICATIONS

This application is a continuation of U.S. patent application Ser. No. 10/443,246, filed May 22, 2003, which claims priority to German Application 102 23 192.3, filed on May 24, 2002, the subject matter of which is incorporated herein by reference.

### BACKGROUND

The present invention relates to devices and methods for the administration or delivery of substances by injection or infusion. More particularly, it relates to ampoules or vials for containing an injectable substance to be dispensed or injected by an administering device such as an infusion pump or an injection device, including syringes or “pen” type devices, and to such administering devices. It further relates to methods of administration or delivery of substances, including medicinal substances, by injection or infusion.

Ampoules are known which can accommodate an injectable substance, such as insulin or other substances, wherein an ampoule filled with a substance to be dispensed is inserted into an infusion pump and is disposed of once the substance contained in it has been dispensed. If the ampoule were not designed as a disposable part, but as a refillable non-disposable part, then costly measures would have to be taken, in particular with regard to the relatively restrictive regulations for the official approval of medical apparatus and instruments, in order to ensure that no contaminants are taken up by the ampoule, for example during a filling process, which could then be injected into a patient.

The administering device required for dispensing the substance from the ampoule is designed, for example in the case of an infusion pump, as a reusable, non-disposable device, since more expensive components, such as control logic or a display, can be used repeatedly without problems and since it would relatively expensive to dispose of these repeatedly usable components after emptying the ampoule once.

### SUMMARY

It is an object of the present invention to provide an ampoule and an administering device into which the ampoule can be inserted, which can meet high quality standards and can be inexpensively and cost-effectively produced together.

In one embodiment, the present invention comprises an ampoule for containing an injectable substance, the ampoule comprising an ampoule body and a component of a means for injecting, the component carried by the ampoule body, wherein other components of the means for injecting are associated with an administering device to which the ampoule can be coupled and are operably coupleable to the component carried by the ampoule body. A method of administering an injectable substance using the ampoule, and the administering device, is encompassed.

In one embodiment, the present invention comprises a method of administering an injectable substance using an ampoule comprising an ampoule body and a component of a means for injecting, the component carried by the ampoule body, wherein other components of the means for injecting are associated with an administering device to which the ampoule can be coupled, the method comprising the steps of providing an injectable substance in the ampoule, coupling the ampoule to the administering device, whereby the component carried by the ampoule body and the other components associated with the administering device are operably coupled, and actuating the means for injecting to administer the injectable substance.

In one embodiment, the present invention comprises an ampoule for containing an injectable substance, the ampoule comprising an ampoule body and at least one component of a means for injecting, the at least one component carried by the ampoule body, wherein at least one other component of the means for injecting is associated with an administering device to which the ampoule can be coupled and is operably coupleable to the at least one component carried by the ampoule body. In some embodiments, the ampoule may carry the entire means for injecting or all or a substantial part of the mechanical aspects of the means for injecting, the administering device comprising a computer or microprocessor type controller for communicating with, operating and/or monitoring the means for injecting.

The ampoule in accordance with the invention, which is suitable for accommodating a substance which can be dispensed via an administering device which can be coupled to the ampoule, such as an infusion pump or injection apparatus, comprises an ampoule body in which the substance to be dispensed or the injectable product can be accommodated. In accordance with the invention, at least one component of a unit required for dispensing the substance, for example a displacing mechanism, is permanently or semi-permanently connectable, connected or attached to the ampoule body, such that an ampoule inserted into an administering device, using said at least one component of the displacing mechanism together with the components of the displacing mechanism provided in the administering device, can be driven or directed such that the substance contained in the ampoule body can be dispensed in doses.

Attaching at least one component of, for example, the displacing mechanism to the ampoule body enables a more cost-effective design of the administering device, since at least this one component of the displacing mechanism, connected to the ampoule body, does not have to be manufactured as an expensive non-disposable part, e.g., made of metal, but can be designed such that it only has to fulfill quality requirements for being used once and, having been used once, can be disposed of together with the ampoule. In this way, components of the administering device previously made of stable and non-abrasive materials, such as suitable metal alloys, can simply be left out, which reduces the cost of the administering device. In addition, it is possible to manufacture said at least one component of the displacing mechanism from a comparatively cheap material, such as a suitable plastic, since such a material is sufficiently reliable to be used once. In some embodiments, the at least one component provided on the ampoule is preferably a mechanical component.

The ampoule can be formed in such a way that a displacing body, for example a stopper, is provided in the ampoule and movable in the ampoule. This stopper can be moved towards the dispensing end of the ampoule by a piston rod connected to the stopper in accordance with the invention, so as to displace a liquid from the interior of the ampoule and thus dispense the injectable substance contained in the ampoule, in doses. In one embodiment, the piston rod can be made of plastic and can be coupled to a suitable drive mechanism of an administering device, e.g., via a thread of a metal threaded rod or other suitable connective structure. In this way, a piston rod no longer has to be provided in the administering device. Once the substance contained in the ampoule has been dispensed, the ampoule can be removed together with the piston rod, and a new ampoule with a new piston rod can be inserted into the administering device. It is therefore also no longer necessary to retract the piston rod which, in previous administering devices, is substantially or completely extended when the ampoule has been emptied, such that using an ampoule in accordance with the invention can save energy and effectively extend the operating life of an administering device. In one embodiment, the piston rod is threaded whereby, when inserting the ampoule, the rod can be screwed onto a threaded rod or other portion of the drive mechanism. In some embodiments, the piston rod comprises multiple portions wherein it is sub-divided along its length or along the length of the threaded portion such that there can be a plug-on, friction or push fit attachment to the drive mechanism, e.g., to a connective structure thereof. In some embodiments, connection may be accomplished manually by the user, in others, the connection between the piston rod and the drive mechanism or the rod of the drive mechanism may be accomplished by the actuation of the drive mechanism. In other words, the user simply places the ampoule in the administering device and, at the time of the initial actuation, the drive mechanism or the drive mechanism rod pushes against the piston rod so as to engage and/or connect to it.

In one embodiment, the threaded rod which engages via threads with the piston rod, which in some embodiments is preferably mounted and/or secured against rotating, is attached to the ampoule together with the piston rod, such that it is also no longer necessary to provide the threaded rod in an administering device, which can effectively further reduce the cost of the administering device.

It should be appreciated that the present invention is not restricted to the mechanism for dispensing a substance from an ampoule as described above. In accordance with the invention, other parts or complete displacing mechanisms may be carried by or attached to the ampoule. For example, in one embodiment, a spring element, such as a spiral spring or a leaf spring, can be attached to the ampoule in accordance with the invention, to exert a constant pressure on the ampoule or a displacing body, such as a stopper movable in the ampoule, whereby a minimum pressure is applied to the substance contained in the ampoule at a dispensing opening of the ampoule. Thus, the substance contained in the ampoule or the product to be dispensed is under pressure generated by the spring element, such that a mechanism does not have to be provided on the administering device to displace the substance. In some embodiments, only a suitable controller, such as a controllable valve, has to be provided on the administering device in order to dispense the desired amount or dose of the substance.

Advantageously, in some embodiments, a dosing or regulating device, for example a controllable valve, can be provided on or associated with the ampoule.

Instead of or in addition to a spring element, other pressure-generating mechanisms can also be provided, for example a pressurized gas container may be used. Such a container, made from a suitable material, would contain or store a pressurized gas for acting on the ampoule, a portion thereof, or a structure associated therewith such that a particular minimum pressure is generated on the substance to be dispensed from the ampoule.

In general, other displacing mechanisms or devices can also be connected to the ampoule, using which the substance contained in the ampoule can be dispensed. For example, an electric drive capable of moving a stopper through a cylindrical ampoule body may be associated with the ampoule.

In embodiments of the present invention requiring power, an energy store is advantageously provided on the ampoule which can store mechanical or electrical energy required for dispensing the substance contained in the ampoule in doses. To this end, a power pack, battery or capacitor can be provided on the ampoule or connected to the ampoule, such that besides the substance to be dispensed contained in it, the ampoule also comprises at least one part which stores the energy required for dispensing the substance in doses. In some embodiments, the energy store associated with the ampoule may be used to power the administering device to dispense the substance from the ampoule. In this case, the administering device no longer has to comprise a power source, for example a battery or battery compartment.

In general, the ampoule can exhibit different forms. It can, for example, be cylindrical and made from a substantially dimensionally stable material, or it could be formed as a pocket, having a flexible portion or being entirely flexible, which can be deformed such that liquid is dispensed from a pocket opening by pressure acting on any point of the outer side of the pocket. The ampoule can be made from various suitable materials, such as plastic, glass, metal or combinations of these.

In accordance with another aspect, the present invention relates to an administering device which can be coupled to an ampoule in accordance with the invention, as described above. The administering device in accordance with the invention correspondingly comprises fewer parts than known administering devices, since, in accordance with the invention, these are provided in or carried on the exchangeable and, in some embodiments, disposable ampoule to be inserted in the administering device. At least one of the following parts could be provided on the ampoule and not on the administering device, depending on the embodiment of the ampoule used in accordance with the invention: a piston rod; a threaded rod; a drive mechanism in general, for moving a displacing body inside or outside the ampoule; a gear for transferring a force or movement from a motor to the ampoule; a motor; a pump; an energy store, such as for example a spring element or an element storing electrical power; a dosing device for dosing the substance to be dispensed; a pressurization device; any other features or structures associated with injection or infusion devices and methods.

Thus, in accordance with the invention, it is possible to permanently or semi-permanently provide only higher-value or higher tolerance components, which can be reused, in the administering device, as non-disposable components, whereas other components can be integrated into or with or carried by the ampoule and exchanged, as disposable components having a more cost-effective design. In some embodiments, the administering device may comprise only a control logic, which is operably coupled, e.g., hard coupled or wirelessly, to an operating unit or operating buttons and a display device and to the ampoule. If, for example, an ampoule in accordance with the invention comprising individual components of a unit required for dispensing is connected to the administering device by suitable means, then a substance can be dispensed in doses through the co-operation of the administering device and the ampoule in accordance with the invention.

### BRIEF DESCRIPTION OF THE DRAWINGS

FIG. 1 is a schematic view of an infusion pump comprising an ampoule, which also may be referred to as an ampoule module, in accordance with the invention; and

FIG. 2 depicts a remote control serving to operate the infusion pump.

### DETAILED DESCRIPTION

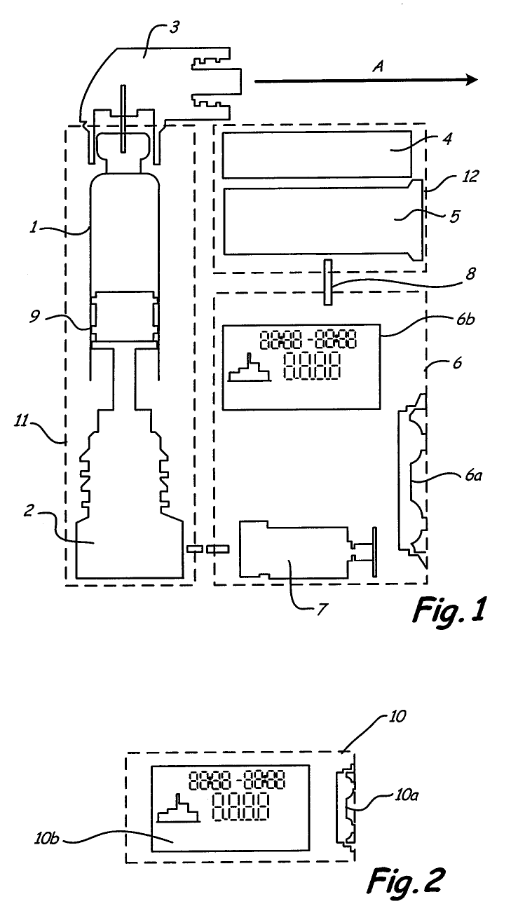

FIG. 1 schematically shows one embodiment of an infusion device, such as an infusion pump, sub-divided into functional groups, and comprising an ampoule module 11 in which an ampoule body 1 is arranged in accordance with the invention. The pump and the module 11 further comprise a suitable drive mechanism 2 for transferring the movement generated by a motor 7 onto a stopper 9 serving to displace an injectable product contained in the ampoule body 1. Suitable drive mechanisms are known in the art of injection and infusion, and typically comprise mechanical and other components sufficient to achieve dispensing of a substance to be dispensed from an ampoule or vial. A movement of the stopper 9 toward the dispensing opening of the ampoule body 1, toward the top of the ampoule body 1 as shown in FIG. 1, results in the injectable substance contained in the ampoule body 1 being dispensed to an infusion set via the adapter 3 coupled to the dispensing opening of the ampoule body 1, in the direction of the arrow A.

In some embodiments, the infusion device can comprise, in a battery and accessories module 12, a sending and/or receiving device 4 for connecting to a remote controller 10 shown by way of example in FIG. 2, and an energy supply 5 such a battery, a power pack or the like.

The battery and accessories module 12 can be connected to a main module 6 via a connection 8, wherein the motor 7, an operating unit 6a, comprising, for example one or more operable keys, and a display 6b can be arranged in said main module 6. Various parameters of the infusion device, such as times and amounts of the substance to be dispensed or other operational variables, can be set via the operating unit 6a, said parameters being used by a suitable controller (not shown) to drive the motor 7 accordingly. In some embodiments, the parameters inputted via the operating unit 6a may be shown on the display 6b together with the operational state of the infusion pump.

FIG. 2 shows a remote control 10 which can optionally be used to operate the infusion pump shown in FIG. 1, such that said infusion pump can be separately carried by or attached to a user and can be operated wirelessly using the remote control 10. The remote control 10 comprises an operating unit 10a which in its design and function can correspond to the operating unit 6a associated with the infusion pump. The remote control 10 comprises a display 10b which can be designed like or reflective of the display 6b of the infusion pump, such that the same settings of the infusion pump can be made using the remote control 10 and the same displays can be shown as via the corresponding operating unit and display of the infusion pump.

It should be noted that, in some embodiments, the assignment of functional sub-assemblies to the individual modules shown in FIG. 1 can also be altered, and that in accordance with the invention, parts of the motor 7, the entire motor 7, the energy supply 5 or parts of it, the operating part 6a, the display 6b or the connection 4 to the remote controller may be arranged in the ampoule module 11, individually or in combination with other elements.

In the foregoing description, embodiments of the invention, including preferred embodiments, have been presented for the purpose of illustration and description. It is not intended to be exhaustive or to limit the invention to the precise forms disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. An ampoule comprising an ampoule body, wherein at least one component of a unit required for dispensing a substance from said ampoule is connected to the ampoule.

2. The ampoule as set forth in claim 1, wherein said ampoule is for use with an administering device for administering an injectable substance.

3. The ampoule as set forth in claim 1, wherein at least one component is a mechanical component.

4. The ampoule as set forth in claim 3, wherein said mechanical component is a piston rod

5. The ampoule as set forth in claim 3, wherein said mechanical component is a threaded rod.

6. The ampoule as set forth in claim 1, wherein a spring element is provided on the ampoule.

7. The ampoule as set forth in claim 6, wherein said spring element is one of a spiral spring or a leaf spring.

8. The ampoule as set forth in claim 1, wherein a pressurized gas container is provided on the ampoule.

9. The ampoule as set forth in claim 1, wherein an electric drive is provided on the ampoule.

10. The ampoule as set forth in claim 1, wherein a dosing device is provided on the ampoule.

11. The ampoule as set forth in claim 1, wherein an energy store is provided on the ampoule.

12. The ampoule as set forth in claim 11, wherein said energy store holds one of electrical or mechanical energy.

13. An administering device, which can be coupled to an ampoule as set forth in claim 1.

14. An ampoule for containing an injectable substance, the ampoule comprising an ampoule body and a component of a means for injecting, the component carried by the ampoule body, wherein another component of the means for injecting is associated with an administering device to which the ampoule can be coupled and is operably coupleable to the component carried by the ampoule body.

15. The ampoule as set forth in claim 14, further comprising a stopper coupled to the ampoule, wherein the component comprises a rod coupled to the stopper.

16. The ampoule as set forth in claim 14, further comprising means for pressurizing the injectable substance.

17. The ampoule as set forth in claim 14, further comprising means for selecting a dose of the injectable substance.

18. The ampoule as set forth in claim 14, wherein the ampoule is disposable.

19. A method of administering an injectable substance using an ampoule comprising an ampoule body and a component of a means for injecting, the component carried by the ampoule body, wherein other components of the means for injecting are associated with an administering device to which the ampoule can be coupled, the method comprising the steps of:
providing an injectable substance in the ampoule;
coupling the ampoule to the administering device, whereby the component carried by the ampoule body and the other components associated with the administering device are operably coupled; and
actuating the means for injecting to administer the injectable substance.

20. The method according to claim 19, wherein the ampoule is disposable.
